(19) United States
(12) Patent Application Publication
(10) Pub. No.: US 2005/0075654 A1
(43) Pub. Date: Apr. 7, 2005

(54) Methods and devices for soft tissue securement

(76) Inventor: Brian Kelleher, San Diego, CA (US)

Correspondence Address: SCOTT EVANS, 1252 COUNTRY HILLS DR., SANTA ANA, CA, 92705, US

(21) Appl. No.: 10/958,100

(22) Filed: Oct. 4, 2004

Related U.S. Application Data
(60) Provisional application No. 60/509,763, filed on Oct. 6, 2003.

(51) Int. Cl. A61F 2/38

(52) U.S. Cl. 606/151

(57) ABSTRACT

Devices and methods for improved soft tissue securement are disclosed, and, in particular, to tissue anchoring elements and deployment thereof. Such tissue anchoring elements may comprise a linkage element and an array of spreading elements. Endoscopic devices and methods are disclosed for deploying multiple anchoring elements to multiple sites and manipulating at least some of the associated linkage elements to approximate selected sites. Applications of such endoscopic devices and methods may include endoluminal therapy such as gastroplasty, which may be used for the treatment of obesity and gastroesophageal disease. Such devices and methods may also include the attachment of a foreign body to a tissue mass. Further aspects of the invention include devices and methods for the modification of mechanical properties of the anchoring sites so as to decrease the likelihood that anchoring elements will pull out. Such modification may include irritating or injuring the tissue within the anchoring sites, thereby causing a healing or scarification response, or may alternatively include deploying a solidifying agent within the anchoring sites.

### PRIORITY INFORMATION

This application claims priority to U.S. Provisional Application No. 60/509763 filed on Oct. 6, 2003, the entire contents of which are hereby expressly incorporated by reference.

### BACKGROUND

1. Field of the Invention

The present invention relates to methods and devices for soft tissue securement, and, in particular, to novel tissue anchoring elements and deployment thereof.

2. Description of the Related Art

The securement of soft tissue segments has traditionally been done using suturing or stapling devices. However, when attaching segments of tissue together that are exposed to tension post-operatively, such techniques often do not hold up over time. For example, in Nissen fundoplication, which is a surgical procedure wherein two segments of the stomach are sewn together, the sutures that hold the segments together are in tension post-operatively. In order to prevent the sutures from pulling through the stomach wall over time, the sites where the sutures puncture the outer wall of the stomach are sometimes reinforced with sections of tear-resistant material, called pledgets.

The use of pledgets is not always possibly, especially when securing the wall of an organ that has a surface not easily accessible during the procedure. As an example, when performing an endoluminal gastroplasty procedure, that is, when sewing the wall of the stomach to itself from within the lumen of the stomach, only the inner wall is accessible. Sutures that are placed through the wall can be strain-relieved with a pledget or similar device only along the inner surface of the wall, but not along the outer wall (unless a pledget or similar device is passed through the wall, which is generally not practical). When sutures placed in this way are exposed to tension, as is the case when a gastroplasty procedure is done to create a gastric restriction, the sutures typically pull out over time.

Similarly, when attaching a foreign body to a segment of soft tissue using attachment techniques such as suturing (without pledgets), if the foreign body is subject to forces postoperatively, the foreign body will typically pull loose from the tissue segment.

There is, therefore, a need for robust tissue securement devices and methods that enable tissue-to-tissue attachment and attachment of foreign bodies to tissue with reduced chance of detachment occurring post-operatively if the securement device is placed under tension. More specifically, there is a need for robust tissue securement devices which can be delivered endoscopically, as through a rigid endoscope, or endoluminally, as through a flexible endoscope.

### SUMMARY

The preferred methods and devices described herein provide for improved methods and devices for tissue securement, and, in particular, to soft tissue anchoring elements and deployment thereof.

In a preferred embodiment of the present invention, a tissue securement system comprises a tissue-penetrating device, an anchoring element and a linkage element. The tissue-penetrating device is deployed at an initial point of securement at least partially through the target tissue mass. The tissue-penetrating device may be an independent element, or it may be part of the anchoring element, or it may be part of a delivery system for the anchoring element. Once the target depth of tissue penetration has been attained, the anchoring element is deployed. The anchoring element preferably incorporates spreading elements to engage a region of tissue wider than the diameter of the tissue-penetrating device. A linkage element is attached to the anchoring element and serves as the part of the system that extends from the initial point of securement to a secondary point of securement. The secondary point of securement may be associated with another tissue segment, another linkage element, or may be associated with a foreign body. The linkage element may be a flexible filament, such as a suture or wire, or may be a length of rigid material.

In at least one preferred embodiment of the invention, the tissue securement system may irritate the tissue so as to trigger a healing response that leads to a toughening or scarification of the tissue in the area of the irritation. The region of scarification is preferably significantly larger than that which may be caused by the deployment of the tissue-penetrating element alone. Such irritation may be carried out prior to, during or after deployment of either the tissue-penetrating device or anchoring element. The anchoring element is preferably positioned within or adjacent to the region of scarification such that the anchoring element will be less likely to pull out than if it was anchored in normal tissue.

In a preferred embodiment of the invention, the anchoring element consists of elements that are deployed from, or are part of, the tissue-penetrating device, and which consist of one or more of the following general categories of elements: hooks, barbs, flanges, mesh, teeth, fingers, whiskers, and the like. Alternatively, the anchoring element may comprise a cluster of semi-rigid tendrils.

In at least one preferred embodiment of the invention, the tissue irritation effect may be created by the deployment of the anchoring element or elements. In a further refinement of this embodiment, the anchoring element may be moved with respect to the tissue mass so as to create an injury within the tissue. Such movement may be accomplished by partial or full rotation of the anchoring element relative to the axis of the tissue-penetrating device, or may be accomplished by repeated advancement and retraction of the anchoring element. During such movement, features on the anchoring element, such as rough or sharp surfaces, barbs or hooks may cause tissue irritation. Such movement and tissue-irritating surfaces may alternatively be associated with the tissue-penetrating device.

In another preferred embodiment of the invention, the tissue may be irritated by thermal means. Such means may include heating, as by heating an element within or adjacent to the tissue, or by the application of energy such as radio frequency (RF) or microwave energy to heat the tissue, or by passing an electric current through the tissue to cause resistive heating. Alternatively, the tissue temperature may be lowered, as by cryogenic freezing. Such thermal irritation may be administered by features within either or both of the anchoring element and the tissue-penetrating device, or by a separate device associated with the system.

In a further preferred embodiment of the invention, the tissue irritation may be accomplished by application of an irritant to the tissue. The irritant may be comprised of one or more of the general classes of substances including sclerosing agents, detergents, cellular toxins and the like, and may be formulated in an appropriate vehicle such as a solution, gel, powder, pellet and the like. The irritant may be injected into a tissue mass, in cases where the anchoring element is to be anchored within the mass, or it may be deposited on the surface of a wall, in cases where the anchoring element is to be anchored against said surface.

In yet another preferred embodiment of the invention, an adhesive agent may be applied to the tissue in order to enhance the securement of the anchoring element in the tissue mass. By way of example, the adhesive agent may be of the general class of instant adhesives known as cyanoacrylates. The adhesive agent may be applied before, during or after deployment of the tissue-penetrating device. Alternatively, the adhesive may be incorporated into the anchoring device and may be triggered by external means such as a temperature change imposed upon the anchoring means, or by a chemical reaction that occurs spontaneously when the adhesive substance reaches body temperature or comes into contact with tissue or associated fluids. In a further refinement of this embodiment, the adhesive agent may also be a tissue irritant, so it not only serves to attach the anchoring element to the tissue, but also to induce scarification of the tissue around the anchoring element.

In a preferred embodiment of the invention, a method is disclosed for deploying at least one anchoring element at a first point of securement and deploying at least one more anchoring element at a second point of securement and linking the two anchoring elements together by at least one linkage element.

In another preferred embodiment of the invention, a method is disclosed for deploying at least one anchoring element at a point of securement in a tissue mass and linking the anchoring element to a foreign body by at least one linkage element.

For purposes of summarizing the preferred embodiments of the invention and the advantages achieved over the prior art, certain objects and advantages have been described herein above. Of course, it is to be understood that not necessarily all such objects or advantages may be achieved in accordance with any particular embodiment of the invention. Thus, for example, those skilled in the art will recognize that the invention may be embodied or carried out in a manner that achieves or optimizes one advantage or group of advantages as taught herein without necessarily achieving other objects or advantages as may be taught or suggested herein.

All of these embodiments are intended to be within the scope of the present invention herein disclosed. These and other embodiments of the present invention will become readily apparent to those skilled in the art from the following detailed description of the preferred embodiments having reference to the attached figures. The invention is not limited to any particular preferred embodiment(s) disclosed.

### BRIEF DESCRIPTION OF DRAWINGS

Having thus summarized the general nature of the invention, certain preferred embodiments and modifications thereof will become apparent to those skilled in the art from the detailed description herein having reference to the figures that follow, of which:

FIG. 1 is a schematic view of a prior art surgical procedure of the stomach showing the use of pledgets;

FIG. 2 is a section view of a stomach wall taken through line 2-2 of FIG. 1, showing a suture anchored with the use of pledgets;

FIG. 3 is a schematic view of a stomach showing a prior art procedure wherein the anterior and posterior walls of the stomach are pulled together using sutures placed endoluminally without pledgets;

FIG. 3a is a section view of the stomach of FIG. 3 taken through line 3a,b-3a,b, showing the stomach walls pulled together with sutures;

FIG. 3b is a section view of the stomach of FIG. 3 taken through line 3a,b-3a,b, showing the stomach walls pulled together with T-anchors;

FIG. 4 is a section view of a tissue securement system;

FIGS. 4a-b are section views of the tissue securement system of FIG. 4 illustrating the steps of penetrating a tissue mass and deploying an anchoring element within the mass;

FIGS. 4c-d are section views of the tissue securement system of FIG. 4 illustrating the steps of penetrating a tissue mass and deploying an anchoring element beyond the mass;

FIG. 5 is a section view of a tissue securement system;

FIG. 5a is a section view showing the tissue securement system of FIG. 5 being deployed into a tissue mass;

FIGS. 5b-d are sections views showing various ways of moving the tissue penetrating device or anchoring element to create an area of tissue irritation or injury;

FIG. 5e is a section view showing the zone of tissue irritation or injury;

FIG. 6 is a section view showing the infusion of an irritating agent or adhesive agent into a tissue mass;

FIG. 6a is a section view showing the tissue mass of FIG. 6 after infusion of the agent and deployment of an anchoring agent;

FIG. 7a is a section view showing the delivery of energy or a temperature gradient to create tissue irritation or injury, wherein the delivery vehicle is the tissue penetrating device;

FIG. 7b is a section view showing the delivery of energy or a temperature gradient to create tissue irritation or injury, wherein the delivery vehicle is the anchoring element;

FIG. 7c is a section view showing the delivery of energy or a temperature gradient to create tissue irritation or injury, wherein the delivery vehicle is a separate delivery device;

FIG. 8 is a perspective view of an anchoring element in its deployed configuration;

FIG. 8a is a section view of the anchoring element of FIG. 8 collapsed into the tissue penetrating device;

FIG. 9 is a perspective view of an anchoring element in its deployed configuration;

FIG. 9a is a section view of the anchoring element of FIG. 9 collapsed into the tissue penetrating device;

FIG. 10 is a perspective view of an anchoring element in its deployed configuration;

FIG. 10a is a section view of the anchoring element of FIG. 10 collapsed into the tissue penetrating device;

FIG. 11 is a perspective view of an anchoring element in its deployed configuration;

4 FIG. 11a is a section view of the anchoring element of FIG. 11 collapsed into the tissue penetrating device;

FIG. 12 is a perspective view of an anchoring element in its deployed configuration;

FIG. 12a is a section view of the anchoring element of FIG. 12 collapsed into the tissue penetrating device;

FIG. 13 is a perspective view of an anchoring element in its deployed configuration;

FIG. 13a is a section view of the anchoring element of FIG. 13 collapsed into the tissue penetrating device;

FIG. 14 is a section view of an anchoring element in its deployed configuration;

FIG. 14a is a section view of the anchoring element of FIG. 14 collapsed into the tissue penetrating device;

FIGS. 15a-c are section views through the wall of a hollow organ showing the placement of anchoring elements at two sites in the wall and the approximation of those sites by bringing the linkage elements of the anchoring elements together;

FIGS. 16a-c are section views through the wall of a hollow organ showing the placement of an anchoring element in the wall and the approximation of a foreign body to the wall by linking the linkage element to the foreign body;

FIG. 17 is a section view of an endoscopic embodiment of the tissue securement system, showing the system traversing the esophagus into the stomach; and

FIG. 18 is a perspective view of the distal portion of the endoscopic embodiment of the tissue securement system of FIG. 17.

### DETAILED DESCRIPTION

The present invention relates to methods and devices for soft tissue securement, and, in particular, to novel anchoring elements and deployment thereof which enable reliable securement of soft tissue to other tissue or to a foreign body.

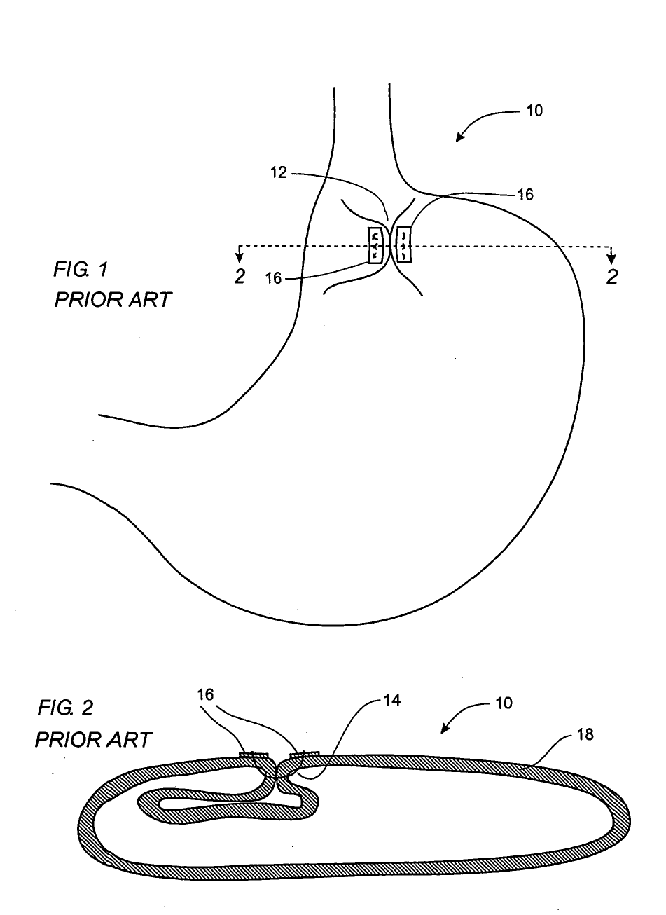

Before describing elements of the present invention, a brief description of prior art devices and methods will be presented. FIG. 1 shows a stomach 10 that has undergone a surgical procedure similar to a Nissen fundoplication, wherein one portion of the stomach is sutured to another portion of the stomach to form tissue securement seam 12. FIG. 2 is a section view taken along line 2-2 in FIG. 1, showing suture 14 passing through stomach wall 18 and pledgets 16. Without pledgets 16, there is a higher likelihood that suture 14 would eventually pull through stomach wall 18, especially when the interface between suture 14 and wall 18 is subjected to post-operative tension or shear force, as is often the case with procedures such as fundoplication. Pledgets 16 provide a strain relief for this interface by distributing the forces at the interface over a greater surface area.

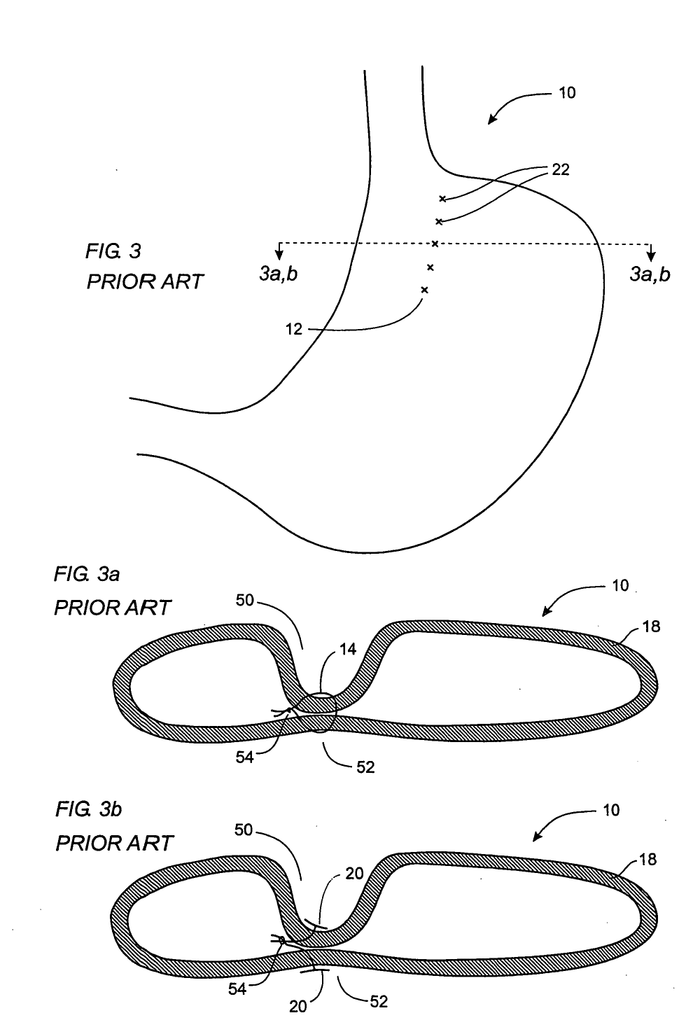

Recent advances in endoscopic instrumentation have enabled the placement of sutures and other securement devices endoscopically. FIG. 3 shows a stomach 10 that has undergone an endoscopic gastroplasty procedure wherein a vertical seam 12 joins the anterior and posterior walls of the stomach. FIGS. 3a and 3b show cross-sections of stomach 10 taken at line 3a, b of FIG. 3, assuming two different types of endoscopic securement. In FIG. 3a, the securement elements are sutures 14, and in FIG. 3b the securement elements are T-anchors 20, each having suture elements 14 extending from them. T-anchors 20 are bar-like elements that typically have a suture connected near their center, and they typically are pushed through tissue in a direction along their long axis, and then the bar-like elements are allowed to pivot relative to the suture so as to anchor within tissue or against a distal wall surface. In FIGS. 3a and 3b, the two sites 50, 52 have been approximated by bringing the ends of sutures 14 together and tying a knot 54. If approximated walls 18 are subjected to post-operative stress along the line of securement, as would be the case if the approximation were intended to create a gastric restriction, sutures 14 would have a high likelihood of pulling through stomach wall 18.

There are at least two reasons why these prior art anchoring devices are susceptible to pulling through the tissue mass in which they are deployed. Both reasons are based on the fact that the prior art anchoring devices have a small surface area along the interface between the device and the tissue in which they are anchored. The first reason is primarily mechanical - - - if the anchoring device is subjected to a high force, it may rip through the tissue, tearing it along the way. The second reason is more physiological - - - the small surface area along the interface causes a high concentration of force at the interface, which can lead to occlusion of blood flow in the tissue along the interface. This occlusion can lead to tissue necrosis, called pressure necrosis, which allows the anchoring element to move through it more easily. As the anchoring element moves through the necrosed tissue, it encounters another layer of healthy tissue and causes a new zone of blood flow occlusion and necrosis to occur. In this way, the prior art anchoring devices can slowly work their way through a relatively large tissue mass, layer by layer.

The present invention is directed at improving over prior art devices and methods by first distributing the forces to which anchoring devices are subjected over a larger surface area or volume of tissue, and second by altering the mechanical properties of the tissue mass in which the anchoring devices are deployed.

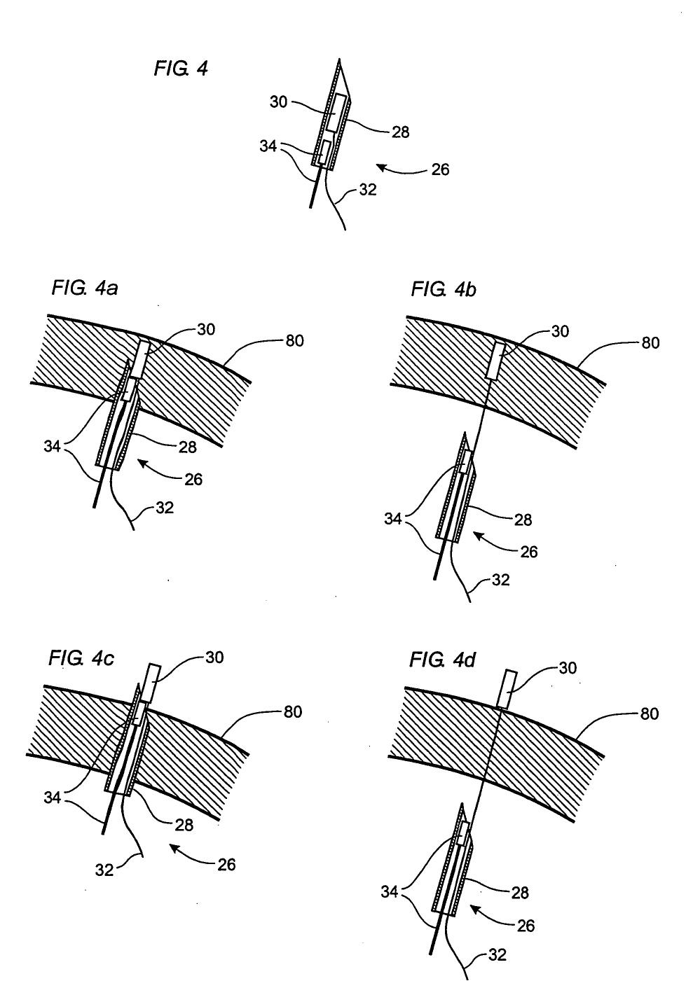

FIG. 4 shows the basic components of a preferred embodiment of the present invention. Tissue securement system 26 comprises tissue-penetrating device 28, anchoring element 30, linkage element 32 and pushing element 34. By way of example, tissue-penetrating device 28 may be a hollow needle made out of a suitable material such as stainless steel, titanium, or the like, and is designed to penetrate the tissue mass of interest. Once tissue-penetrating device 28 has reached the desired depth of penetration, as shown in FIG. 4a, pushing element 34 is advanced relative to tissue-penetrating device 28 to deploy anchoring element 30 into tissue mass 80. As shown in FIG. 4b, after deployment of anchoring element 30, pushing element 34 and tissue-penetrating device 28 are retracted from tissue mass 80, without pulling in linkage element 32. Linkage element 32 may be a suture or wire made from materials known to those in the art. Alternatively, linkage element 32 may be a loop or other form, including rigid forms, designed to engage another object or linking element. FIGS. 4c and 4d are analogous to FIGS. 4a and 4b, except anchoring element 30 is shown being deployed beyond the outer surface of tissue mass 80. It will be appreciated that the anchoring element 30 depicted in FIGS. 4, 4a-d is not intended to be descriptive other than in its relation to the other elements of tissue securement system 26. Details of preferred embodiments of anchoring element 30 are discussed below.

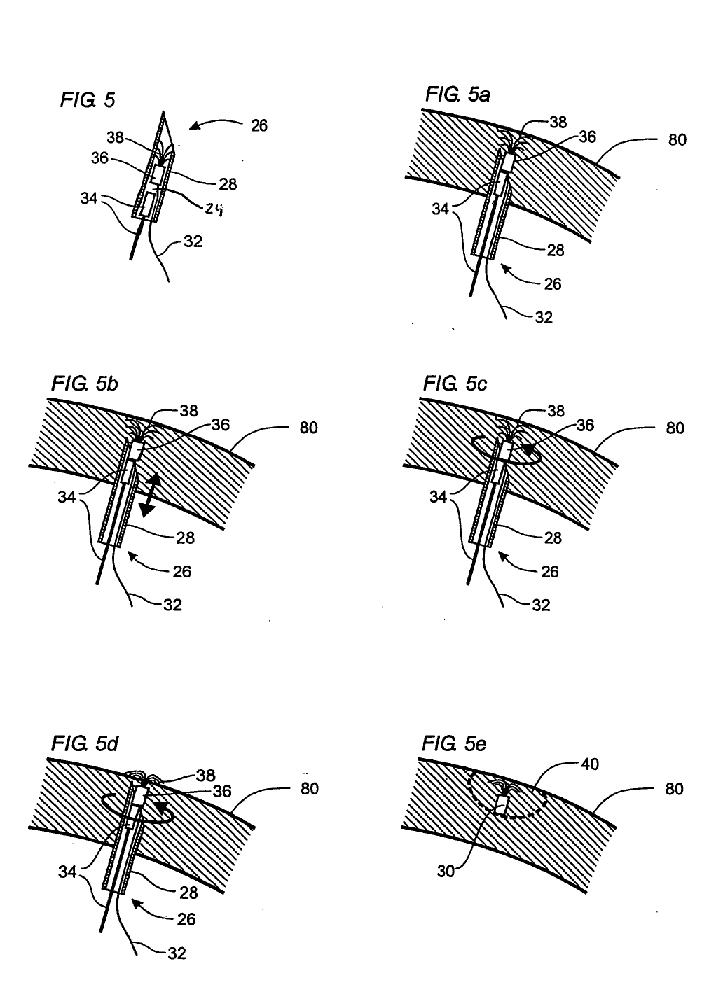

FIG. 5 depicts tissue securement system 26 showing more details of anchoring element 30, including base 36 and spreading elements 38. Base 36 captures spreading elements 38 and linkage element 32. Note that base 36 may not be needed if a direct connection between spreading elements 38 and linkage element 32 is established. In the embodiment shown, when anchoring element 30 is confined inside the inner lumen 29 of tissue-penetrating device 28, spreading elements 38 are straightened along the axis of the lumen 29 of tissue-penetrating device 28. In FIG. 5a, tissue securement system 26 is shown advanced into tissue mass 80, wherein spreading elements 38 have been allowed to spread out. It will be appreciated that a pulling force along the axis of linkage element 32 will be translated to spreading elements 38, and that such force will be distributed over a greater volume of tissue and a larger surface area than an equivalent force applied to a prior art suture or T-anchor. Therefore, the likelihood of anchoring element 30 pulling out of tissue mass 80 should be significantly lower than for an analogous suture or T-anchor.

Preferred embodiments for altering the composition of the tissue surrounding the anchoring element 30 will now be disclosed. FIGS. 5b and 5c show various ways in which the tissue in the region of deployment of anchoring element 30 may be injured or at least irritated by mechanical movement of parts of tissue securement system 26. By injuring or irritating the tissue, a healing effect will likely be triggered in the affected tissue, which will preferably lead to changes in the composition of the tissue making it less susceptible to having anchoring element 30 pull out. Such changes may include scarification of the tissue, which may be associated with increased fibrosis and decreased vascularity. Increased fibrosis may increase the mechanical strength of the tissue, while decreased vascularity may reduce the possibility of forces on an anchoring element in the tissue causing pressure necrosis.

In FIG. 5b, the irritating or injury effect is created by moving tissue-penetrating device 28 back and forth axially, preferably with anchoring element 30 at least partially deployed, such that the back and forth motion causes spreading elements 38 to move into and out of the surrounding tissue. Adding elements such as barbs, hooks, teeth, rough edges or points along the surfaces of spreading elements 38 or other portions of tissue securement system 26 may enhance the injurious effect caused by this motion. It will be appreciated that such movement may be accomplished by moving spreading elements 38, tissue-penetrating device 28, pushing element 34 or linkage element 32, or some combination thereof.

FIG. 5c shows the rotation of elements of tissue securement system 26 to injure or at least irritate surrounding tissue. Preferably anchoring element 30 is rotatably linked to tissue-penetrating device 28. Alternatively, anchoring element 30 may be rotated independently of tissue-penetrating device 28. Pushing element 34 or linkage element 32 may also be linked to the rotational movement. Such rotation may be full rotation or partial back-and-forth rotation. It will be apparent that multiple rotating elements may be incorporated into the design, and that such multiple rotating elements may have differing directions of rotation in order to enhance the injurious effect and minimize the tendency for driving elements to “wind up” during rotational movement. Additionally, certain rotating elements may rotate so as to cut tissue against a fixed or counter-rotating element of the system. It will be appreciated that a separate element or elements may by used to create the injurious effect, rather than employing features of tissue securement system 26, as depicted in the above figures.

FIG. 5d shows anchoring element 30 deployed substantially outside the wall of tissue mass 80, such that spreading elements 38 are in contact with the outer surface of tissue mass 80. In this configuration, movement of elements of tissue securement system 26, such as the rotational motion shown, may thereby cause tissue injury or irritation to the surface of tissue mass 80. In this mode of operation, it is preferable that the deployment portion of tissue securement system 26, as well as the configuration of spreading elements 38, be optimized so as to minimize the chance of injury tissue or organs surrounding tissue mass 80.

FIG. 5e shows the zone of irritation or injury 40 caused by the various mechanical actions described above. Also shown is anchoring element 30 deployed into zone 40. Zone 40 may be within tissue mass 80 or it may be on a surface of tissue mass 80.

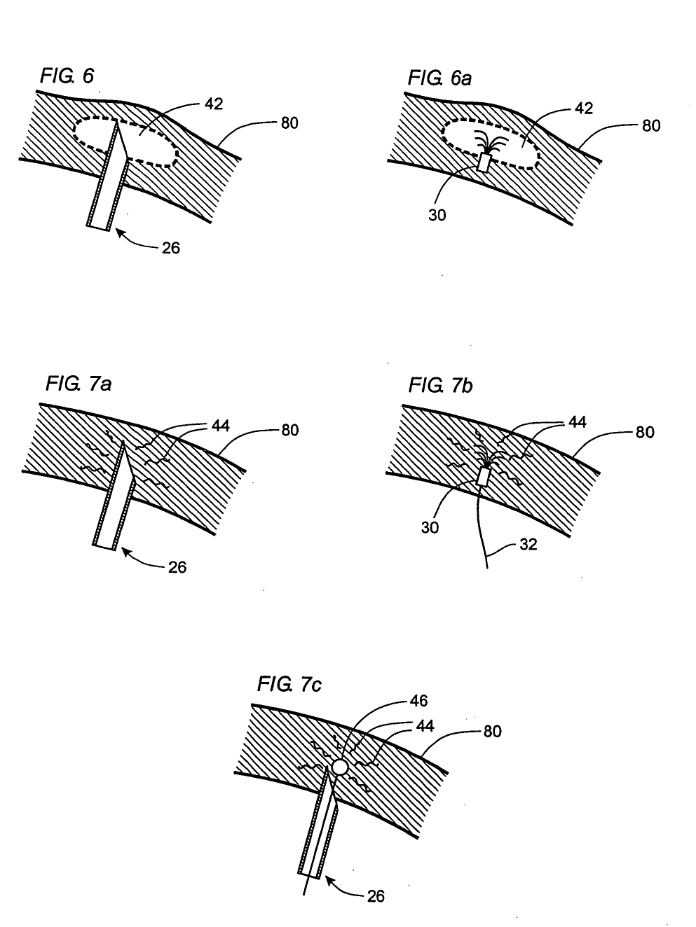

FIG. 6 shows a different preferred embodiment for modifying the properties of the region into which anchoring element 30 is deployed. In one preferred embodiment, an irritant is deployed into or onto the tissue. The irritant may be a sclerosing agent, detergent, cellular toxin or the like, and may be formulated as a solution, gel, powder, pellet or the like. In an alternative preferred embodiment, a solidifying agent such as a cyanoacrylate may be deployed into or onto the tissue. In FIG. 6, a volume of agent 42 is shown injected into tissue mass. In FIG. 6a, anchoring element 30 is depicted as having been deployed into the volume of agent 42. In the case where agent 42 is an irritant, agent 42 will preferably be quickly absorbed by, or diffused into, tissue mass 80, such that deployment of anchoring element 30 will be into tissue and not solely into agent 42. In the case where agent 42 is a solidifying agent, preferably solidification does not occur until anchoring element 30 is deployed. Such solidification of agent 42 may be controlled by formulation, or by use of a secondary agent that catalyzes solidification. Whether agent 42 is an irritant or a solidifying agent, it will be appreciated that anchoring element 30 may be deployed into tissue mass 80 before, during or after deployment of agent 42.

FIGS. 7a-c show various preferred embodiments in which anchoring element 30 is deployed into tissue that is modified by the application of energy or by a change in temperature. In FIG. 7a, tissue-penetrating device 28 is shown as the conduit for such energy or temperature change, as indicated by lines 44. In the case where the energy is delivered as electricity, tissue-penetrating device 28 may function as either a monopolar or bipolar electrode, transmitting electricity through the target tissue. Such electricity may either be in the form of direct current or alternating current. Embodiments based on alternating current may utilize a high-frequency source, such as a radio-frequency generator, thereby inducing thermal injury similar to electrocautery. In embodiments where the energy is heat, tissue-penetrating device 28 may incorporate a heating element (not shown), or may be a conduit for heat generated by an adjacent heating element. Similarly, tissue-penetrating device 28 may serve as a means for lowering the temperature of the target tissue, as by cryogenic freezing. Tissue-penetrating device 28 may also serve as an antenna to provide microwave energy to surrounding tissue, thereby causing heating and injury.

FIG. 7b shows anchoring element 30 serving as the energy or temperature conduit, in which case linkage element 32 may serve to provide electrical energy in certain embodiments requiring electricity. FIG. 7c shows a separate element 46 that is placed into tissue mass 80 to create the injurious effect.

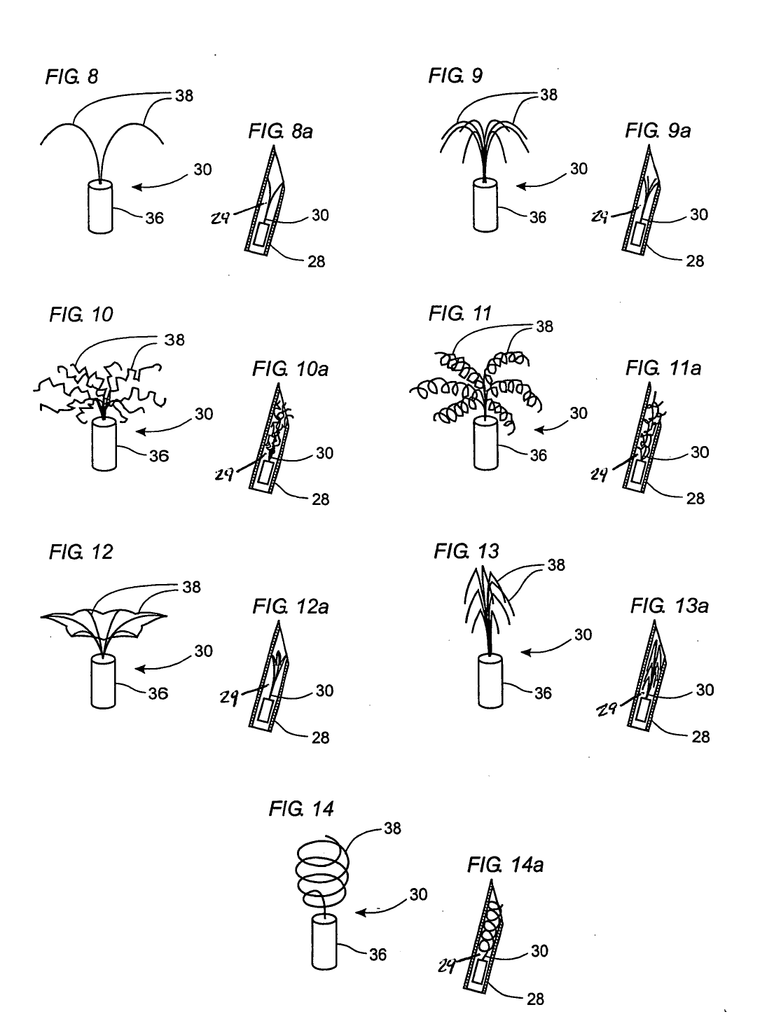

FIG. 8 shows a specific configuration for anchoring element 30 in which spreading elements 38 comprise substantially flat elements. As shown in FIG. 8a, when anchoring element 30 is loaded into the hollow needle embodiment of tissue-penetrating device 28, spreading elements 38 flatten out along the long axis of the lumen 29 of tissue-penetrating device 28. Spreading elements 38 may be formed from a shape memory metal such as Nitinol.

FIG. 9 shows another specific configuration for anchoring element 30 in which spreading elements 38 comprise an array of curved elements that deploy in a radially spaced fashion to infiltrate much of the volume of tissue around anchoring element 30. As shown in FIG. 9a, when anchoring element 30 is loaded into the hollow needle embodiment of tissue-penetrating device 28, spreading elements 38 straighten out along the long axis of the lumen 29 of tissue-penetrating device 28. Spreading elements 38 may be formed from metals such as stainless steel, or a shape memory metal such as Nitinol.

FIG. 10 shows yet another configuration for anchoring element 30 in which spreading elements 38 comprise an array of randomly twisted and angled tendrils that push through and engage the tissue during deployment of anchoring element 30. As shown in FIG. 10a, when anchoring element 30 is loaded into the hollow needle embodiment of tissue-penetrating device 28, spreading elements 38 flatten out along the long axis of the lumen 29 of tissue-penetrating device 28. Spreading elements 38 may be formed from metals such as stainless steel, or a shape memory metal such as Nitinol. In the case where anchoring element 30 is deployed into a bolus of solidifying agent 42, such as that depicted in FIG. 6a, spreading elements 38 may be made from a polymer or fibrous material which will become encased when agent 42 solidifies.

FIG. 11 shows an alternative configuration for anchoring element 30 in which spreading elements 38 comprise an array of helical wires that helically engage the tissue during deployment of anchoring element 30. As shown in FIG. 11a, when anchoring element 30 is loaded into the hollow needle embodiment of tissue-penetrating device 28, spreading elements 38 flatten out along the long axis of the lumen 29 of tissue-penetrating device 28. Spreading elements 38 may be formed from a shape memory metal such as Nitinol. In the case where anchoring element 30 is deployed into a bolus of solidifying agent 42, such as that depicted in FIG. 6a, spreading elements 38 may be made from a polymer or fibrous material which will become encased when agent 42 solidifies.

FIG. 12 shows still another configuration for anchoring element 30 in which spreading elements 38 comprise a fluted wireform that opens up when anchoring element 30 is deployed. As shown in FIG. 12a, when anchoring element 30 is loaded into the hollow needle embodiment of tissue-penetrating device 28, spreading elements 38 flattens out along the long axis of the lumen 29 of tissue-penetrating device 28. Spreading elements 38 may be formed from metals such as stainless steel, or a shape memory metal such as Nitinol. In the case where anchoring element 30 is deployed into a bolus of solidifying agent 42, such as that depicted in FIG. 6a, spreading elements 38 may be made from a polymer or fibrous material which will become encased when agent 42 solidifies.

FIG. 13 shows yet another configuration for anchoring element 30 in which spreading elements 38 comprise an array of bent, barb-like tendrils. As shown in FIG. 13a, when anchoring element 30 is loaded into the hollow needle embodiment of tissue-penetrating device 28, spreading elements 38 are bent along the long axis of the lumen 29 of tissue-penetrating device 28, and then spring out as shown in FIG. 13. Spreading elements 38 may be formed from metals such as stainless steel, or a shape memory metal such as Nitinol.

FIG. 14 shows still another configuration for anchoring element 30 in which spreading element 38 comprises a helical wireform. As shown in FIG. 14a, when anchoring element 30 is loaded into the hollow needle embodiment of tissue-penetrating device 28, spreading element 38 is stretched along the long axis of the lumen 29 of tissue-penetrating device 28. Spreading element 38 may be formed from metals such as stainless steel, or a shape memory metal such as Nitinol.

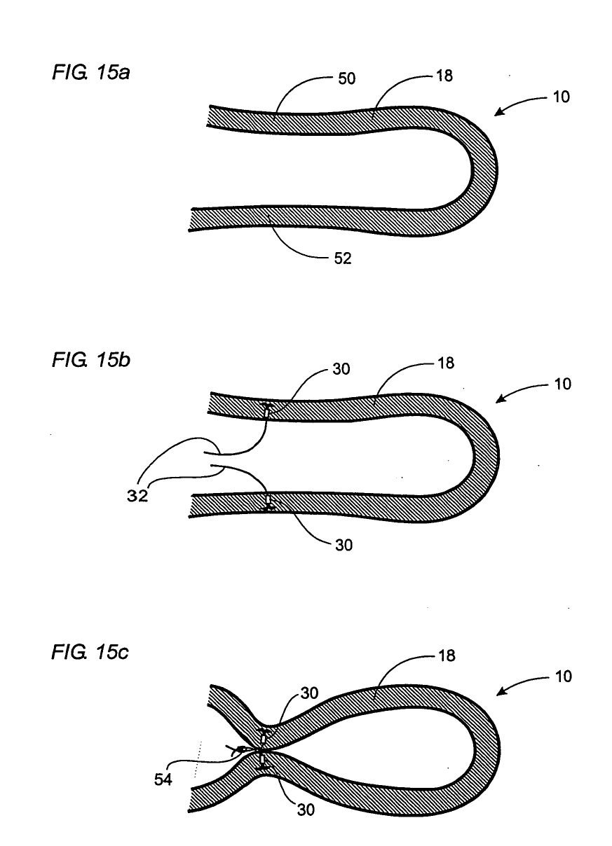

FIGS. 15a-c depict a preferred method for approximating two tissue masses, such as the walls of a hollow organ like the stomach. FIG. 15a shows two sites 50, 52 of stomach 10 having a wall 18. FIG. 15b shows anchoring elements 30, each having linkage element 32, having been placed at sites 50, 52 using the methods described previously. FIG. 15c shows the two linkage elements 32 associated with anchoring elements 30 having been brought together and secured with knot or securing element 54, thereby approximating sites 50, 52 of stomach wall 18.

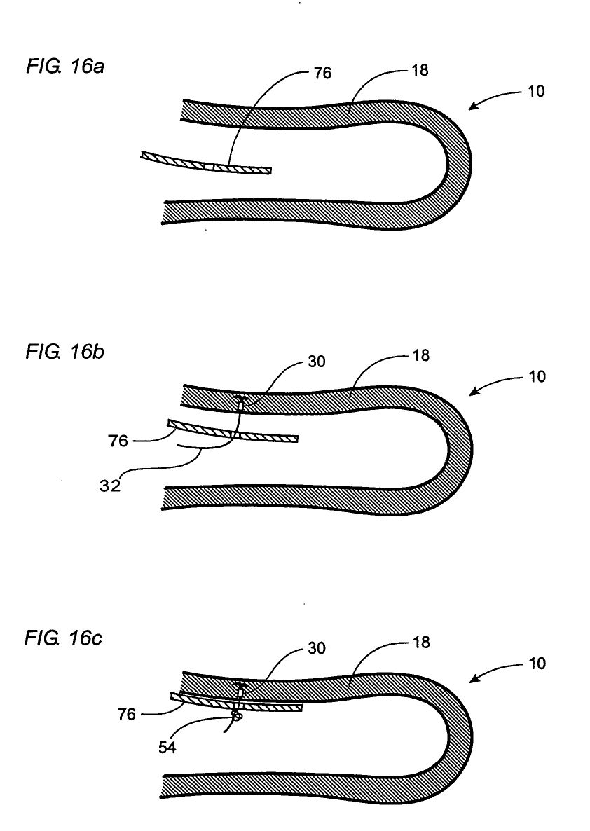

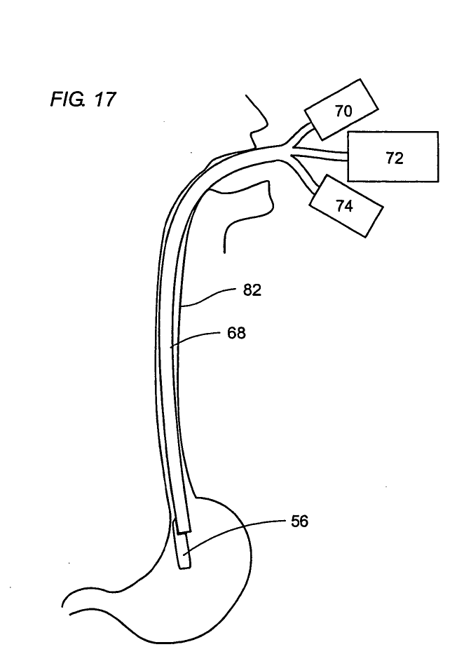

FIGS. 16a-b depict a preferred method for attaching a foreign body to a tissue mass such as a stomach wall using the devices and methods described previously. In FIG. 16a, foreign body 76 is depicted adjacent to wall 18 of stomach 10. In FIG. 16b, anchoring element 30 is shown deployed into wall 18 with linkage element being threaded through, or otherwise coupling with, foreign body 76. In FIG. 16c, foreign body 76 is shown in close approximation to wall 18, with linkage element 32 having been tied or anchored with knot or securing element 54.

FIG. 17 shows an endoscopic embodiment of tissue securement system 26. Endoscope 68 is shown traversing the esophagus 82 from the mouth to the stomach 10. Associated with the distal end of the system is an endoscopic accessory 56 that deploys tissue anchoring elements. Associated with the proximal end of endoscope 68 are a set of endoscopic controls 72, which may comprise steering knobs and valves for air, water and suction, a set of accessory controls 74 to activate mechanisms within endoscopic accessory 56, and a linkage management means 70, which allows for the handling of linkage elements from multiple anchoring elements deployed by the accessory.

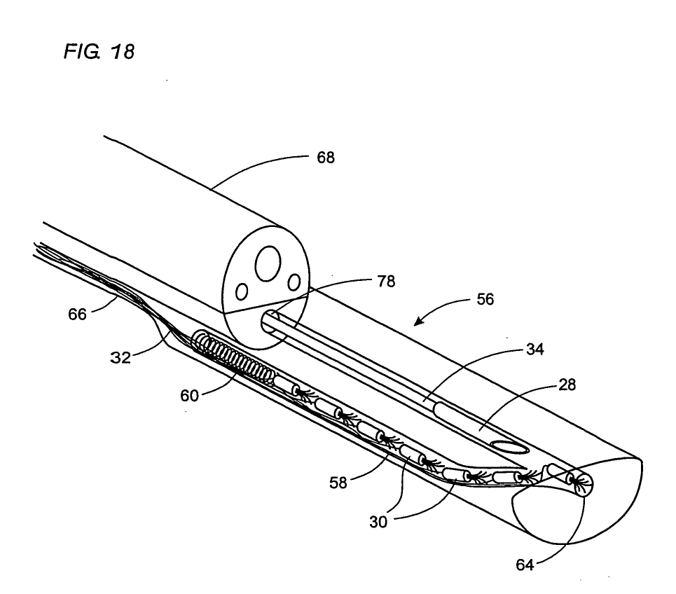

FIG. 18 shows a closer view of the tip of endoscope 68 and endoscopic accessory 56 depicted in FIG. 17. Endoscopic accessory 56 is preferably capable of deploying one or more anchoring elements 30 to selected sites. By way of example, accessory 56 may carry a payload 58 of anchoring elements 30, which may be urged distally by spring 60 along the payload path until such path merges with output channel 64. Once an anchoring element 30 is loaded in output channel 64, tissue penetrating device 28 is advanced by pushing element 34 so that tissue penetrating device 28 enshrouds anchoring element 30 and then delivers it out channel 64 into a desired tissue target. Advancement of tissue penetrating device 28 and pushing element 34 may be triggered by a set of push-pull mechanisms that extend through working channel 78, or alongside endoscope 68, to accessory control block 74. Element 34 preferably consists of a coaxial push-pull mechanism wherein a central wire is surrounded by a coiled sheath. To advance tissue-penetrating device 28, both the central wire and outer sheath are advanced together. To push the anchoring element out of tissue-penetrating device 28, just the central wire is advanced. Accessory control block 74 may also include a mechanism for rotating one or more elements of tissue securement system 26, such as anchoring element 30, to injure or irritate the tissue.

After anchoring element 30 is deployed, element 34 is pulled back to retract tissue-penetrating device 28 thereby clearing the output channel. When output channel 64 becomes clear, another anchoring element 30 is urged into channel 64 as a result of the force of spring 60. Linkage elements 32 from anchoring elements 30 may be allowed to extend freely alongside of endoscope 68, or they may be contained in conduit 66. Linkage management means 70 may allow for proximal or distal knot tying and knot pushing, or may include means for enabling the proximal or distal deployment of securement elements as a substitute for knots. Such securement elements may take the form of crimpable lengths of metal tubing, for example.

It will be appreciated that the timing of events associated with the securement methods described herein may be altered to maximize the durability of the anchoring sites. By way of example, certain methods described thus far imply deployment of anchoring elements at multiple sites followed relatively immediately by approximation of such sites by linking the linkage elements associated with each site. However, it may be advantageous to first deploy anchoring elements to desired sites and then at a later point approximate such sites by bringing the anchoring sites together, thereby allowing the tissue to react to any irritation or injury and thus strengthen the anchoring site before it is subjected to forces.

Although certain embodiments and examples have been described herein, it will be understood by those skilled in the art that many aspects of the methods and devices shown and described in the present disclosure may be combined differently and/or modified to form still further embodiments. Additionally, it will be recognized that the methods described herein may be practiced using any device suitable for performing the recited steps. Such alternative embodiments and/or uses of the methods and devices described above and obvious modifications and equivalents thereof are intended to be within the scope of the present disclosure. Thus, it is intended that the scope of the present invention should not be limited by the particular embodiments described above, but should be determined only by a fair reading of the claims that follow.

1. A method for soft tissue securement including the steps of modifying the properties of the tissue at or near an anchoring site and deploying a first anchoring element at said site.

2. The method of claim 1 wherein said modification of tissue includes irritating or injuring the tissue.

3. The method of claim 2 wherein said injury or irritation induces scarring in said tissue.

4. The method of claim 2 wherein said injury or irritation is accomplished by heating said tissue.

5. The method of claim 4 wherein said heating is accomplished by heating a heating element near said tissue.

6. The method of claim 4 wherein said heating is accomplished by passing electric current through said tissue.

7. The method of claim 4 wherein said heating is accomplished by applying radio frequency or microwave energy to said tissue.

8. The method of claim 2 wherein said injury or irritation is accomplished by freezing said tissue.

9. The method of claim 2 wherein said injury or irritation is accomplished by chemical means.

10. The method of claim 9 wherein said chemical means includes the application of a sclerosing agent, detergent or cellular toxin.

11. The method of claim 2 wherein said injury or irritation is accomplished by mechanical agitation of said tissue.

12. The method of claim 11 wherein said mechanical agitation is caused by movement of a moveable element relative to said tissue.

13. The method of claim 12 wherein said moveable element is said first anchoring element.

14. The method of claim 13 wherein said movement includes full or partial axial or rotational movement of at least part of said first anchoring element.

15. The method of claim 2 wherein said injury or irritation is caused by the application of radiant, conductive or thermal energy through or by said anchoring element.

16. The method of claim 1 wherein said first anchoring element is deployed at a first anchoring site, said first anchoring element having a first linkage element.

17. The method of claim 16 wherein a second anchoring element is deployed at a second anchoring site, said second anchoring element having a second linkage element, wherein said first and second anchoring sites are brought together by bringing said first and second linkage elements together.

18. The method of claim 16 wherein a foreign body is approximated to said first anchoring site by linking said first linkage element to said foreign body.

19. The method of claim 2 wherein said first anchoring element includes an array of spreading elements, said spreading elements having a first state and a second state.

20. The method of claim 19 wherein the transition of said spreading elements from said first state to said second state causes at least part of said irritation or injury.

21. A method for soft tissue securement including the steps of deploying a solidifying agent at or near an anchoring site and deploying an anchoring element at said site.

22. The method of claim 21 wherein said solidifying agent is a substance which changes from a substantially liquid or gel phase to a substantially solid phase after deployment in the tissue.

23. The method of claim 22 wherein said solidifying agent is a gluing agent.

24. The method of claim 23 wherein said gluing agent is of the general classification of cyanoacrylates.

25. An apparatus for securing tissue comprising a delivery device and an anchoring element, said anchoring element having spreading elements and a linkage element, said anchoring element having a long axis, said spreading elements having a first state and a second state wherein in said first state said spreading elements are substantially aligned along said long axis and in said second state said spreading elements are deployed in substantially more than one plane relative to said long axis.

26. The apparatus of claim 25 wherein said delivery device comprises a tissue-penetrating device.

27. The apparatus of claim 26 wherein said tissue penetrating device is a hollow needle and said anchoring element is delivered at least partially through the lumen of said hollow needle.

28. The apparatus of claim 25 wherein the transition of said spreading elements from said first state to said second state is caused by the movement of said anchoring element with respect to said delivery device.

29. The apparatus of claim 25 wherein said spreading elements comprise shapes of at least one of the following: wireforms, hooks, barbs, flanges, mesh, teeth, fingers, whiskers, tendrils or helixes.

30. The apparatus of claim 25 wherein said spreading elements comprise one or more helical forms which helically engage said tissue at said anchoring site during said transition from said first state to said second state.

31. An apparatus for securing tissue comprising an anchoring element and means for modifying the mechanical properties of tissue within or adjacent to an anchoring site.

32. The apparatus of claim 31 wherein said means for modifying the mechanical properties includes means for injuring or irritating said tissue within or adjacent to said anchoring site.

33. The apparatus of claim 32 wherein said means for injuring or irritating said tissue includes moveable elements that mechanically agitate said tissue.

34. The apparatus of claim 32 wherein said means for injuring or irritating said tissue includes elements that apply radiant, conductive or thermal energy to said tissue.

35. The apparatus of claim 32 wherein said means for injuring or irritating said tissue includes elements that deploy an irritating agent.

36. The apparatus of claim 35 wherein said irritating agent may be at least one of the following: a sclerosing agent, detergent or a cellular toxin.

37. The apparatus of claim 31 wherein said anchoring element comprises spreading elements and a linkage element.

38. The apparatus of claim 37 wherein said anchoring element is moveable with respect to said tissue, and wherein movement of said anchoring element causes injury or irritation of said tissue.

39. The apparatus of claim 38 wherein said anchoring element has a long axis, said spreading elements have a first state and second state wherein in said first state said spreading elements are substantially aligned along said long axis and in said second state said spreading elements are deployed in substantially more than one plane relative to said long axis.

40. The apparatus of claim 39 wherein the transition of said spreading elements from said first state to said second state causes at least a portion of said injury or irritation of said tissue.

41. The apparatus of claim 37 wherein said linkage element may be used to secure said anchoring site to a second site or to a foreign body.

42. The apparatus of claim 41 wherein said linkage element is a filament such as a thread, suture, wire, or loop.

43. The apparatus of claim 31 wherein said modification of said tissue is accomplished by deploying a solidifying agent into said tissue.

44. An endoscopic device for tissue securement comprising a payload containing at least one tissue anchoring element and means for deploying said at least one anchoring element into a tissue mass, wherein said at least one anchoring element has a long axis and includes spreading elements and a linkage element, wherein said spreading elements have a first state in which said spreading elements are substantially constrained along said long axis and a second state wherein said spreading elements are substantially unconstrained and spread out in more than one plane relative to said long axis, wherein the deployment of said anchoring element causes the transition from said first state to said second state.

45. The apparatus of claim 44 wherein said endoscopic device further comprises means for modifying the mechanical properties of a zone within or adjacent to an anchoring site.

46. The apparatus of claim 45 wherein said means for modifying the mechanical properties includes means for injuring or irritating the tissue in said zone.

47. The apparatus of claim 45 wherein said means for modifying the mechanical properties includes means for deploying a solidifying agent into said zone.

48. The apparatus of claim 44 including means for sequentially deploying multiple anchoring elements to multiple sites and further comprising means for bringing the linkage elements from at least a portion of said anchoring elements together to approximate at least a portion of said multiple sites.

49. A method for performing endoluminal tissue securement in an organ using an endoscopic device capable of deploying one or more anchoring elements, each anchoring element including a linkage element and an array of spreading elements, wherein said spreading elements have a first state in which said spreading elements are substantially constrained and a second state wherein said spreading elements are substantially unconstrained, wherein the deployment of each anchoring element causes the transition from said first state to said second state, comprising the steps of deploying multiple anchoring elements to multiple sites and manipulating the linkage elements from selected anchoring elements so as to approximate a portion of said multiple sites.

50. The method of claim 49 wherein the organ comprises the stomach.

51. The method of claim 50 wherein said approximated sites comprise segments of the stomach wall.

52. The method of claim 51 whereby said approximation of segments of the stomach wall is for the treatment of obesity.

53. The method of claim 51 whereby said approximation of segments of the stomach wall is for the treatment of gastroesophageal reflux disease.

54. A method of placing an anchoring element in a soft tissue mass comprising a first step of modifying the mechanical properties of a selected zone within or on the surface of said tissue mass and a second step of deploying an anchoring element within or adjacent to said zone.

55. The method of claim 54 wherein said modification of the mechanical properties comprises injuring or irritating the tissue within said zone and the time between said first and second step is proportional to the response time of the tissue to said injury or irritation.

56. The method of claim 54 wherein said modification of the mechanical properties comprises deploying a solidifying agent within said zone and the time between said first and second steps is proportional to the solidification time of said solidifying agent.
